(19) United States
(12) Patent Application Publication
(10) Pub. No.: US 2015/0127074 A1
(43) Pub. Date: May 7, 2015

(54) DEVICE FOR EXTERNAL MEDICAL TREATMENT USING LIGHT WITH SEVERAL SERIES OF INCREASING PULSE FREQUENCIES

(71) Applicant: Rolf Thiberg, Akersberga (SE)

(72) Inventor: Rolf Thiberg, Akersberga (SE)

(73) Assignee: BIOLIGHT PATENT HOLDING AB, Stockholm (SE)

(21) Appl. No.: 14/397,608

(22) PCT Filed: Apr. 26, 2013
(86) PCT No.: PCT/SE2013/050468

§ 371 Date: Oct. 28, 2014

(30) Foreign Application Priority Data
May 7, 2012 (SE) 1250462-7

(51) Int. Cl. A61N 5/06

(52) U.S. Cl. CPC A61N 5/06; A61N 2005/0651; A61N 2005/0626; A61N 2005/0659; A61N 2005/0663
USPC 607/90

(57) ABSTRACT

Device for external medical treatment using light, including a light emitting element arranged to abut against or near the body of an individual and a drive device for the light emitting element, which includes light emitting diodes. The drive device controls the light emitting element to emit light of one or several wavelengths during one or several predetermined times and to emit the light in a pulsating manner. The light emitting element includes a cover and a light emitting diode plate, supporting the light emitting diodes positioned thereon with a certain distance between two adjacent light emitting diodes. The device emits light in two or several series with increasing pulse frequencies F1, F2, Fn, where F1 is a fundamental frequency and where F2 and Fn are multiples of the fundamental frequency. The device emits a pulse repetition frequency that varies about the respective frequency F1, F2 and Fn.

The present invention relates to a device for external medical light treatment.

In for instance the Swedish patent no 502 784, a device for external medical treatment using light is disclosed, comprising a light emitting means arranged to abut against or be held near the body of an individual, as well as a drive device for the light emitting means, which light emitting means comprises light emitting diodes or the corresponding, and is arranged to emit infrared light. The invention according to the said patent specifies that the drive device is arranged to control the light emitting means to, in a first step, emit infrared light during a first predetermined time, and to thereafter, in a second step, emit visible light during a second predetermined time, and that the drive device is arranged to control the light emitting means to emit the infrared light and the visible light, respectively, in a pulsating manner according to a predetermined series of pulse frequencies.

In the international application WO 95/19810, a device is disclosed wherein the light emitting means emits light according to two or several series of gradually increasing pulse frequencies.

It is also known to also emit other monochromatic lights for treatment of various medical conditions.

Furthermore, it has turned out that treatment with only one or several monochromatic lights and other than infrared lights, such as visible light of various colours, which is emitted according to a certain pulse frequency, results in very good treatment results.

It has turned out that a device of the said type very successfully can be used for treatment of many medical conditions and injuries, for example athletic injuries, strains, muscular pain, joint pains, headaches, various inflammatory conditions, various skin disorders such as acne, back pains, etc., under the condition that the lights are emitted in a certain way. Hereby, the light treatment has a positive effect on the healing process for injuries, and it alleviates and/or cures various illnesses.

The device has also very successfully been used for treatment of wounds, for example of diabetic foot wounds and within geriatric care, and also within aesthetics and equestrian sports.

Hence, there is a knowledge that light treatment through the emission of a certain light in certain frequency series results in a substantially increased effect in the form of shortening of the time for the curing or alleviation of an illness.

Since the light treatment is presumed to have effect already at the cell level, it is desirable to use a dynamic light image and that the whole area on the body to be treated receives an equal amount of light over time.

A problem with light treatment is that all persons or animals that are treated do not respond in the same way to certain parameters, such as pulse frequencies during emission of pulsating light, something that should be a consequence of the uniqueness of each individual. Thus, it has turned out that a modification of various parameters can result in improved treatment results for certain individuals. However, it is difficult to know how the parameters are to be set in order to better suit a certain individual.

The present invention solves this problem.

Hence, the present invention relates to a device for external medical treatment using light, comprising a light emitting means arranged to abut against or be held near the body of an individual as well as a drive device for the light emitting means, which light emitting means comprises light emitting diodes or the corresponding, which light emitting diodes are arranged to emit light in different wavelength intervals, wherein the drive device is arranged to control the light emitting means to emit light of one or several wavelengths during one or several predetermined times and to emit said light in a pulsating manner, which light emitting means comprises a cover and a light emitting diode plate, supporting the light emitting diodes, wherein the light emitting diodes are positioned on said plate so that there is a certain distance between two adjacent light emitting diodes, and wherein the device is arranged to emit light in two or several series with increasing pulse frequencies F1, F2, Fn, where F1 is a fundamental frequency and where F2 and Fn are multiples of the fundamental frequency, and is characterised in that the device is arranged to emit a pulse repetition frequency that varies about the respective frequency F1, F2 and Fn.

Below, an embodiment of the invention is described, partly with reference to the following drawings, where

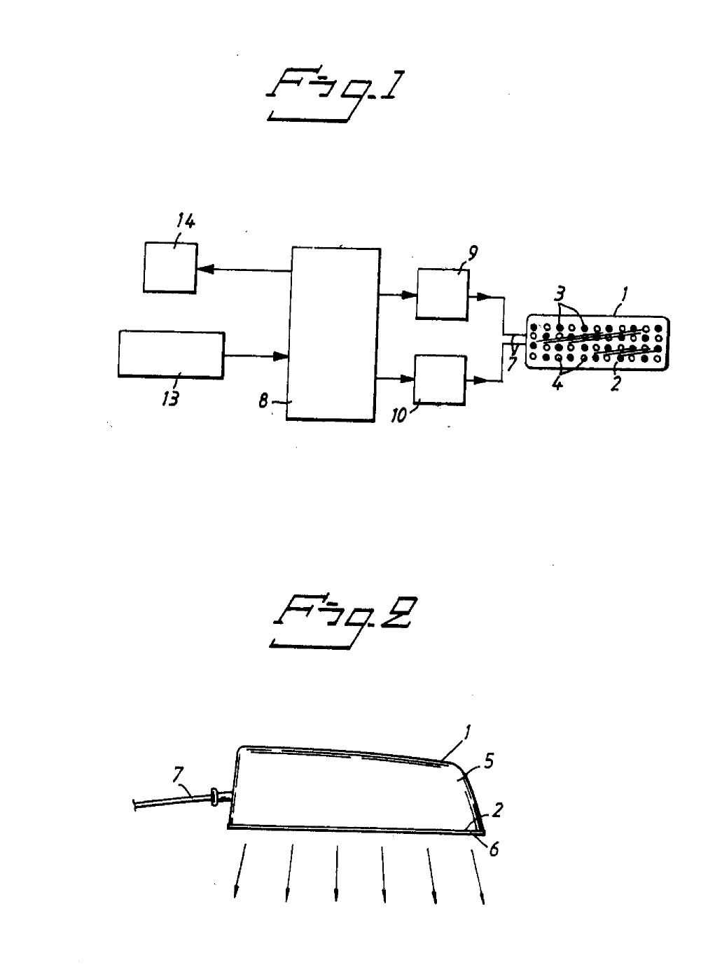

FIG. 1 schematically shows a block diagram of a device according to the invention,

FIG. 2 shows a light emitting means according to an embodiment of the invention in a side view.

FIG. 1 shows, generally, a device for external medical treatment with light, comprising a light emitting means 1 arranged to abut against or be held close to the body of an individual. The light emitting means comprises a housing 1 arranged with a transparent pane 2. Under this pane 2, inside the housing, there is a number of light emitting diodes 3, 4, see FIG. 1. The light emitting diodes 3, 4 are arranged to emit light through the pane 2 when supplied with current via a cable 7. During use, the housing 1 is held so that the pane 2 abuts against or is close to the body part in question, of for example a person to be treated. For instance, there may be a wound.

Furthermore, the device comprises a drive device 9, 10 for the light emitting means, see the block scheme in FIG. 1, wherein the light emitting means 1 is also shown from below. The drive device is arranged to control the light emitting means 1 to emit different monochromatic light of different wavelengths and in a pulsating manner, according to a predetermined pulse frequency or series of pulse frequencies.

The light emitting means 1 comprises light emitting diodes 3, 4, arranged to emit a substantially monochromatic, visible light in any of the colours violet, blue, yellow, orange, red or green, as well as infrared and other non-visible wavelengths. Which of these lights to be used depends on the illness or type of injury to be treated.

The light emitting means 1 may comprise a type of light emitting diodes 3 which are arranged to emit for instance infrared light. These are marked using filled circles in FIG. 1. Visible light may be emitted using other light emitting diodes 4, which are marked using unfilled circles in FIG. 1. The light emitting diodes for infrared light and visible light, respectively, are preferably semiconductors of the type GaAs (Gallium Arsenide). The light emitting diodes can also be arranged to emit light of other wavelengths. Thus, the light emitting means 1 may comprise light emitting diodes arranged to emit an essentially monochromatic, visible light in any of the colours violet, blue, yellow, orange, red or green, as well as infrared and other non-visible wavelengths.

The drive device for the light emitting means comprises, in a way which is known as such, a computer 8 for controlling the drive circuits 9, 10, which drive circuits are fed with signals from the computer and in turn drive the light emitting diodes via the conductor 7. To the drive device or the computer, a keyboard 13 is connected, using which the operator can key in data in order to thereby control the drive device to control the light emitting means in a desired way. Suitably, there is also a display 14 for showing the settings input via the keyboard 13.

According to the invention, the device is arranged to emit a pulse repetition frequency that varies about the respective frequency F1, F2 and Fn.

Preferably, the light pulses are square pulses.

Hereby, for each pulse repetition frequency with its variation, some frequencies will suit certain individuals, while some other pulse repetition frequencies will suit other individuals at the same time as all pulse repetition frequencies affect essentially all individuals to an effective but varying degree.

Preferably, a series of frequencies is emitted, after which a number of additional series with the same frequencies and starting from the frequency F1 are emitted.

According to a preferred embodiment, the said variation is between 1% and 10% of the respective frequency.

According to another preferred embodiment, the device is arranged so that the fundamental frequency F1 is the fundamental frequency according to Schumann resonances. Schumann resonances are extremely low frequencies of the earth's electromagnetic field. Schumann resonances are global electromagnetic resonances, arising by lightning discharges excited in the cavity formed by the earth surface and the ionosphere.

As an example, a series for wound healing can be F1=12 Hz+/−1 Hz, F2=24 Hz+/−2 Hz, F3 72 Hz+/−4 Hz, F5=780 Hz+/−176 Hz, F6=8556+/−32 Hz and F7=9900 Hz+/−64 Hz.

It is preferred that the device is arranged to first emit infrared light during a predetermined first time, and thereafter visible red light during a second predetermined time.

Above, a number of embodiments have been described. However, the design of the light emitting device may be varied, as well as the said variation. Therefore, the present invention shall not be limited to the above disclosed embodiments, but may be varied within the scope of the enclosed claims.

1. Device for external medical treatment using light, comprising a light emitting means (1) arranged to abut against or be held near the body of an individual as well as a drive device (9,10) for the light emitting means, which light emitting means comprises light emitting diodes or the corresponding (3,4), which light emitting diodes are arranged to emit light in different wavelength intervals, wherein the drive device (9,10) is arranged to control the light emitting means (1) to emit light of one or several wavelengths during one or several predetermined times and to emit said light in a pulsating manner, which light emitting means comprises a cover and a light emitting diode plate (2), supporting the light emitting diodes, wherein the light emitting diodes are positioned on said plate so that there is a certain distance between two adjacent light emitting diodes, and wherein the device is arranged to emit light in two or several series with increasing pulse frequencies F1, F2, Fn, where F1 is a fundamental frequency and where F2 and Fn are multiples of the fundamental frequency, characterised in that the device is arranged to emit a pulse repetition frequency that varies about the respective frequency F1, F2 and Fn.

2. Device according to claim 1, characterised in that the variation is between 1% and 10% of the respective frequency.

3. Device according to claim 1, characterised in that the device is arranged so that the fundamental frequency F1 is the fundamental frequency according to Schumann resonances.

4. Device according to claim 1, characterised in that the device is arranged to first emit infrared light during a predetermined first time and thereafter visible red light during a second predetermined time.

5. Device according to claim 2, characterised in that the device is arranged so that the fundamental frequency F1 is the fundamental frequency according to Schumann resonances.

6. Device according to claim 2, characterised in that the device is arranged so that the fundamental frequency F1 is the fundamental frequency according to Schumann resonances.

7. Device according to claim 3, characterised in that the device is arranged so that the fundamental frequency F1 is the fundamental frequency according to Schumann resonances.
